(19) United States
(12) Patent Application Publication
(10) Pub. No.: US 2014/0249372 A1
(43) Pub. Date: Sep. 4, 2014

(54) RIGID-ENDOSCOPE OVERSHEATH

(71) Applicant: FUJIFILM CORPORATION, Tokyo (JP)

(72) Inventors: Koji YOSHIDA, Ashigarakami-gun (JP); Nobuyuki TORISAWA, Ashigarakami-gun (JP)

(73) Assignee: FUJIFILM CORPORATION, Tokyo (JP)

(21) Appl. No.: 14/274,333

(22) Filed: May 9, 2014

Related U.S. Application Data
(62) Division of application No. 13/469,014, filed on May 10, 2012.

(30) Foreign Application Priority Data
Jun. 9, 2011 (JP) 2011-128857

(51) Int. Cl. A61B 1/12; A61B 1/00

(52) U.S. Cl. CPC A61B 1/126; A61B 1/00135
USPC 600/121

(57) ABSTRACT

A rigid-endoscope oversheath having a leading end, a base end, a longitudinal shaft, and a lumen into which a rigid endoscope can be inserted, the oversheath including a flow path through which fluid flows from the base end toward the leading end along the longitudinal shaft, an ejection port formed in a same plane with a leading end plane of the rigid-endoscope oversheath to eject the fluid flowing through the flow path from the leading end plane of the flow path, a peripheral portion of the port being at least partially composed of an elastic member deformable by the fluid flowing through the flow path, and a valve at least partially attached to a leading end of the flow path to openably close the leading end of the flow path.

The present application is a Divisional Application of U.S. patent application Ser. No. 13/469,014, filed on May 10, 2012, which is based on and claims priority from Japanese patent application No. 2011-128857, filed on Jun. 9, 2011, the entire contents of which is incorporated herein by reference.

### BACKGROUND OF THE INVENTION

1. Field of the Invention

The present invention relates to a rigid-endoscope oversheath.

2. Description of the Related Art

During surgery and/or treatment within body cavities using a rigid endoscope, the glass face at the leading end of the rigid endoscope may get dirty with splattered blood and/or body fluid. Further, using an electrosurgical knife or the like may generate smoke and/or mist, which in turn may attach to the glass face of the rigid endoscope. When the leading end face of the rigid endoscope gets dirty, the rigid endoscope is taken out of the body cavity and extraneous matters on the leading end face of the rigid endoscope are removed with, for example, gauze to ensure the scope and visibility.

There have been proposed some techniques of covering an endoscope with an endoscope cleaning sheath to clean the leading end face of the endoscope (see Japanese Unexamined Patent Application Publication No. Hei 8-173370, for example), providing a cleaning nozzle at the leading end face of a rigid endoscope to clean the cover glass of the rigid endoscope (see Japanese Unexamined Patent Application Publication No. Hei 5-207962, for example), and forming a nozzle on a cover of a head in which the leading end portion of an air-supply tube is housed (see Japanese Unexamined Patent Application Publication No. Hei 4-146717, for example). There have further been proposed techniques of forming a shape-memory-alloy air/fluid-supply nozzle on the leading end part of an endoscope (see Japanese Unexamined Patent Application Publication No. Sho 61-36718, for example) and making an endoscope less likely to be cooled even when cold water may flow through a pipe line in a sheath (see Japanese Unexamined Patent Application Publication No. Hei 9-135804, for example), as well as an endoscope cleanable without increasing the diameter of its portion to be inserted into a body (see Japanese Unexamined Patent Application Publication No. 2003-220018, for example).

However, the techniques described in Japanese Unexamined Patent Application Publication Nos. Hei 8-173370, Hei 5-207962, Hei 4-146717, Hei 9-135804, and 2003-220018 include no change in the shape of the ejection port, resulting in that the angle and direction of ejection of cleaning solution such as saline and/or gas such as carbon dioxide cannot be changed. Also, the technique described in Japanese Unexamined Patent Application Publication No. Sho 61-36718 uses a shape-memory-alloy nozzle, which requires temperature change to be deformed. Using an endoscope within a body cavity may cause the leading end of the endoscope to have a temperature higher than that of the body due to heat from an illumination source, which may result in a deformation of the nozzle shortly after the start of the use of the endoscope independently of the temperature of the cleaning solution. This may also result in that a desired wide area of the leading end face of the endoscope cannot be cleaned.

### SUMMARY OF THE INVENTION

It is hence an object of the present invention to make adjustable the angle of ejection of fluid at a leading end face of a rigid endoscope by adjusting the flow rate of fluid flowing through a flow path in a rigid-endoscope oversheath.

A first aspect is directed to a rigid-endoscope oversheath having a leading end, a base end, a longitudinal shaft, and a rumen into which a rigid endoscope can be inserted, the oversheath including: a flow path through which fluid (gas such as air and/or liquid such as cleaning solution) flows from the base end toward the leading end along the longitudinal shaft; and an ejection port formed in a leading end portion of the rigid-endoscope oversheath to eject therefrom the fluid flowing through the flow path (the ejection port, through which fluid flows out of the rigid-endoscope oversheath, facing the leading end of the rigid-endoscope oversheath), a peripheral portion of the port being at least partially composed of an elastic member deformable by the fluid flowing through the flow path.

In accordance with the first aspect above, the ejection port is formed in the leading end portion of the rigid-endoscope oversheath to eject therefrom fluid flowing through the flow path from the base end toward the leading end along the longitudinal shaft of the rigid-endoscope oversheath. A peripheral portion of the ejection port is at least partially formed with an elastic member deformable by the fluid flowing through the flow path. Since the peripheral portion of the ejection port is at least partially formed with the elastic member, the ejection port is deformed elastically, when fluid is ejected from the ejection port, according to the flow rate of the fluid. The elastic deformation of the elastic member results in an increase in the size of the ejection port. Since the ejection port is deformed according to the amount of ejection of the fluid, the direction of ejection of the fluid from the ejection port also changes. That is, the direction of ejection of the fluid can be controlled according to the flow rate of the fluid flowing through the flow path. For example, when the flow rate is low, the ejection port is not deformed to remain narrower, so that the fluid is ejected at higher speed. This allows the fluid with even a lower flow rate to be ejected far away in the direction in which the ejection port is opened. When the flow rate is high, the ejection port is deformed to be widened, so that the fluid can be ejected widely. In particular, since this aspect includes no adjustment of the temperature of the fluid, there is no need to raise and keep the temperature of the cleaning solution and/or gas flowing through the flow path higher than that of the body, which allows the direction of ejection of the fluid to be adjusted relatively easily.

The peripheral portion of the ejection port is surrounded by, for example, a soft portion deformable by the fluid flowing through the flow path and a hard portion rigid against the fluid.

The ejection port may be connected with the flow path and formed in a leading end portion of a nozzle facing the center of a leading end face of the rigid-endoscope oversheath.

The hard portion of the ejection port may be fixed with respect to the rigid-endoscope oversheath or may be displaceable with respect to the oversheath.

The upper surface of the nozzle (approximately parallel to a lens surface formed in a leading end face of a rigid endoscope when the rigid endoscope is inserted in the rumen of the rigid-endoscope oversheath) may constitute a relatively soft portion while the side surface of the nozzle (approximately perpendicular to a lens surface formed in a leading end face of a rigid endoscope when the rigid endoscope is inserted in the rumen of the rigid-endoscope oversheath) may constitute a relatively hard portion or the upper surface of the nozzle may constitute a relatively hard portion while the side surface of the nozzle may constitute a relatively soft portion.

The nozzle is composed of, for example, an elastic member.

The rigid-endoscope oversheath may further include a valve at least partially attached to a leading end of the flow path to openably close the leading end of the flow path.

A portion through which the valve is attached to the leading end of the flow path or the valve itself may be composed of the elastic member.

The valve may include first and second valves with one end thereof being attached to a wall surface of the flow path at the leading end of the flow path, and the first and second valves may be configured to openably close the leading end of the flow path.

The first valve may be provided on the outer peripheral side at a leading end face of the rigid-endoscope oversheath, while the second valve may be provided on the inner peripheral side at the leading end face of the rigid-endoscope oversheath, and the first valve may be softer than the second valve.

The rigid-endoscope oversheath may further include a cap to be put on the leading end of the rigid-endoscope oversheath, and the ejection port may be formed in the cap.

A second aspect is directed to a rigid-endoscope oversheath having a leading end, a base end, a longitudinal shaft, and a rumen into which a rigid endoscope can be inserted, the oversheath including: a flow path through which fluid flows from the base end toward the leading end along the longitudinal shaft; and an ejection port formed in a leading end portion of the rigid-endoscope oversheath to eject therefrom the fluid flowing through the flow path, in which the ejection port includes a movable portion displaceable with respect to the rigid-endoscope oversheath and a non-movable portion fixed with respect to the rigid-endoscope oversheath, and the movable portion is urged such that a first area of the ejection port when fluid is ejected from the ejection port is greater than a second area of the ejection port when no fluid is ejected from the ejection port.

In accordance with the second aspect above, the ejection port includes the movable portion displaceable with respect to the rigid-endoscope oversheath and the non-movable portion fixed with respect to the rigid-endoscope oversheath, and the movable portion is urged such that the first area of the ejection port when fluid is ejected from the ejection port is greater than the second area of the ejection port when no fluid is ejected from the ejection port. Since the ejection port is widened according to the amount of ejection of the fluid, the direction of ejection of the fluid from the ejection port also changes. That is, also in this second aspect, the direction of ejection of the fluid can be controlled according to the flow rate of the fluid flowing through the flow path.

### BRIEF DESCRIPTION OF THE DRAWINGS

FIG. 1 is a perspective view showing a rigid endoscope and a rigid-endoscope oversheath.

FIG. 2 is a cross-sectional view of the rigid endoscope covered with the rigid-endoscope oversheath.

FIG. 3 is a perspective view of the rigid endoscope covered with the rigid-endoscope oversheath.

FIG. 4 is a perspective view of a cleaning nozzle.

FIG. 5 is a perspective view of the cleaning nozzle with an ejection port being deformed.

FIGS. 6A to 6C show the development of deformation of the ejection port according to the flow rate.

FIG. 7 is a perspective view of a cleaning nozzle with an ejection port being deformed.

FIGS. 8A and 8B show aspects where fluid is ejected from the cleaning nozzle.

FIGS. 9 and 10 are cross-sectional views of a rigid endoscope covered with a rigid-endoscope oversheath.

FIG. 11A is a cross-sectional view of a rigid endoscope covered with a rigid-endoscope oversheath and FIG. 11B is a cross-sectional view in the vicinity of an ejection port.

FIG. 12 is a perspective view of a rigid endoscope covered with a rigid-endoscope oversheath.

FIG. 13 is a cross-sectional view in the vicinity of an ejection port.

FIG. 14 is a perspective view of a rigid endoscope covered with a rigid-endoscope oversheath.

FIG. 15 is a cross-sectional view in the vicinity of an ejection port.

FIG. 16 is a perspective view showing a leading end of an insertion portion of a rigid endoscope and a cap put on the insertion portion.

FIG. 17 is a cross-sectional view of the cap put on the insertion portion of the rigid endoscope.

FIG. 18 is a cross-sectional view of the rigid endoscope covered with a rigid-endoscope oversheath with the cap put thereon.

### DESCRIPTION OF THE PREFERRED EMBODIMENTS

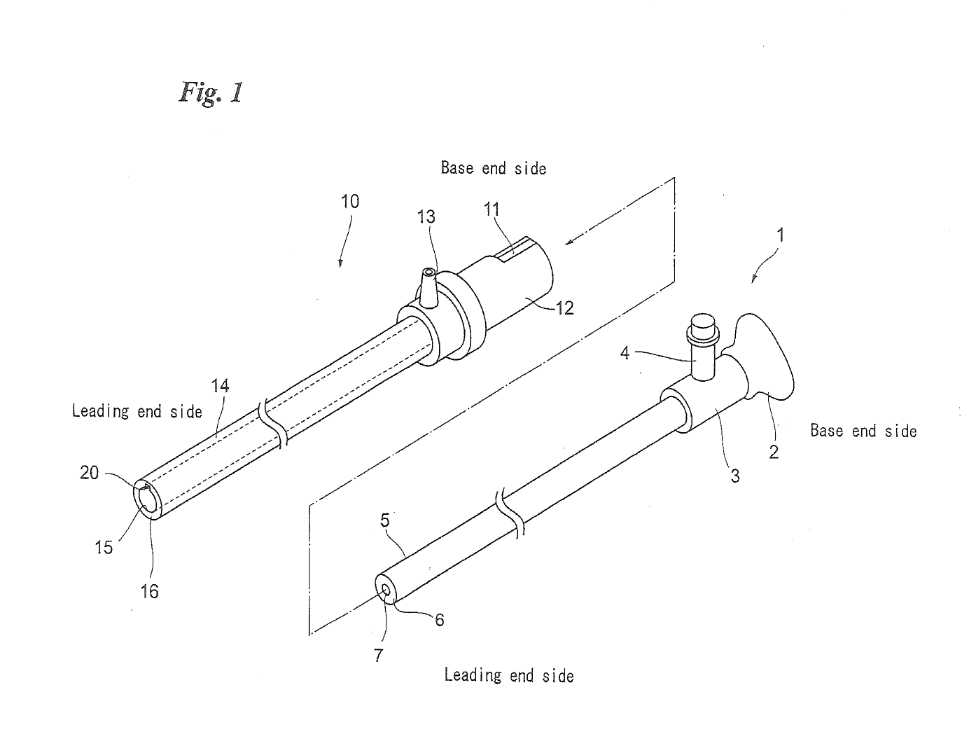

FIG. 1 is a perspective view showing a rigid endoscope 1 and a rigid-endoscope oversheath 10 to cover the rigid endoscope 1, according to a preferred embodiment of the present invention.

The rigid endoscope 1 includes a relatively long cylindrical insertion portion 5 to be inserted into a body cavity. At the base end of the insertion portion 5 is formed an operational portion (gripper) 3. At the rear end (base end) of the operational portion 3 is formed an eye piece 2. On the side surface of the operational portion 3 is formed a light-guide base 4 in a radially standing manner. The light-guide base 4 is intended to receive a light guide (not shown) for illuminating a test object. At the leading end 6 of the insertion portion 5 is attached a cover glass 7.

The rigid-endoscope oversheath 10 is formed with a circular tubular insertion portion 14 to cover the insertion portion 5 of the rigid endoscope 1. Inside the insertion portion 14 is formed an insertion path 15 for receiving the insertion portion 5 of the rigid endoscope 1 therethrough. At the base end of the insertion portion 14 is formed an attachment 12. On the periphery of the attachment 12 is formed a guide groove 11 for arranging the light-guide base 4 of the rigid endoscope 1 in a predetermined position. On the periphery of the attachment 12 is also formed a cleaning base 13 in a radially standing manner. At the leading end face 16 of the insertion portion 14 is formed a cleaning nozzle 20.

The rigid endoscope 1 is covered with the rigid-endoscope oversheath 10 and a tube (not shown) is connected to the cleaning base 13. Cleaning solution flows through the tube and then a flow path (not shown in FIG. 1) in the rigid-endoscope oversheath 10 to be ejected from the cleaning nozzle 20. The cleaning solution ejected from the cleaning nozzle 20 cleans the cover glass 7.

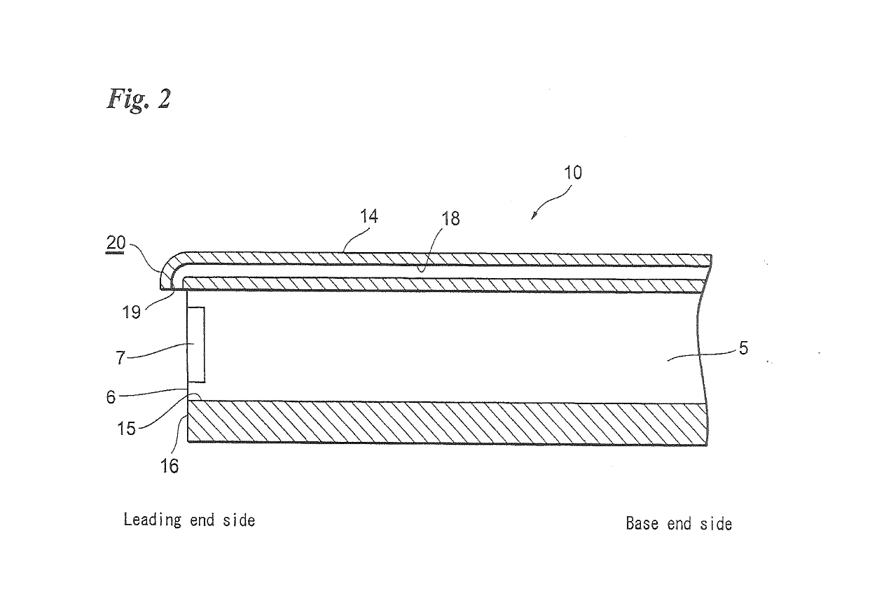

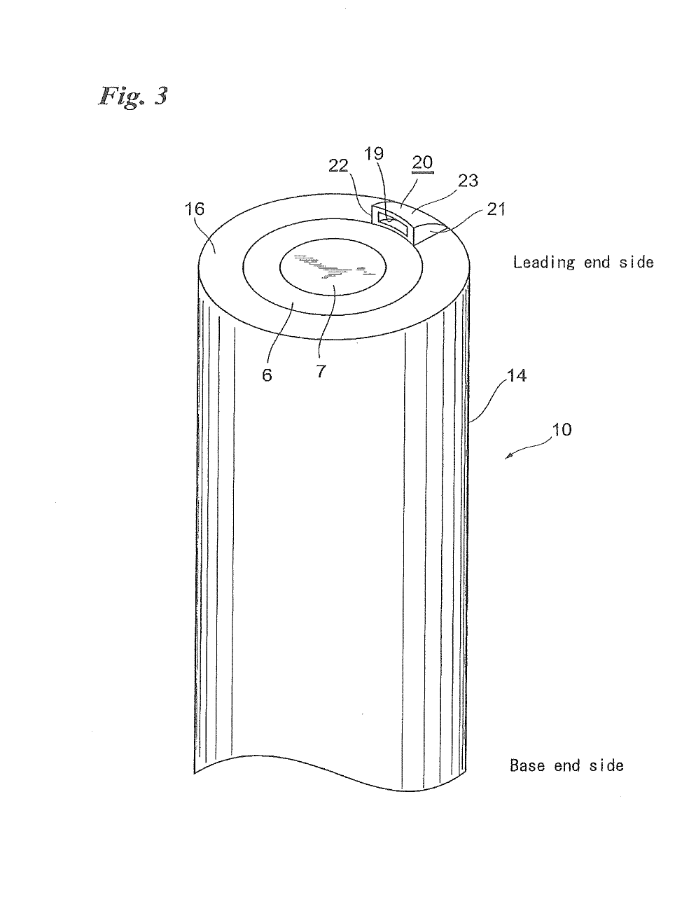

FIG. 2 is a longitudinally-cutaway partial side cross-sectional view of the rigid endoscope 1 covered with the rigid-endoscope oversheath 10. FIG. 3 is a perspective view of the rigid endoscope 1 covered with the rigid-endoscope oversheath 10. In FIG. 3, the rigid endoscope 1 is drawn vertically with the leading end 6 thereof being arranged on the upper side.

When the insertion portion 5 of the rigid endoscope 1 is inserted in the insertion path (rumen) 15 of the rigid-endoscope oversheath 10 so as to be covered with the rigid-endoscope oversheath 10, the leading end face 6 of the rigid endoscope 1 and the leading end face of the rigid-endoscope oversheath 10 lie on approximately the same plane.

The insertion portion 14 of the rigid-endoscope oversheath 10 has a longitudinal shaft (not shown), and a flow path 18 through which fluid such as cleaning solution and/or air flows from the base end toward the leading end is formed longitudinally in the insertion portion 14. As mentioned above, the cleaning nozzle 20 is formed at the leading end face 16 of the insertion portion 14. The cleaning nozzle 20 faces the cover glass 7, that is, the center of the leading end face 16 of the rigid-endoscope oversheath 10.

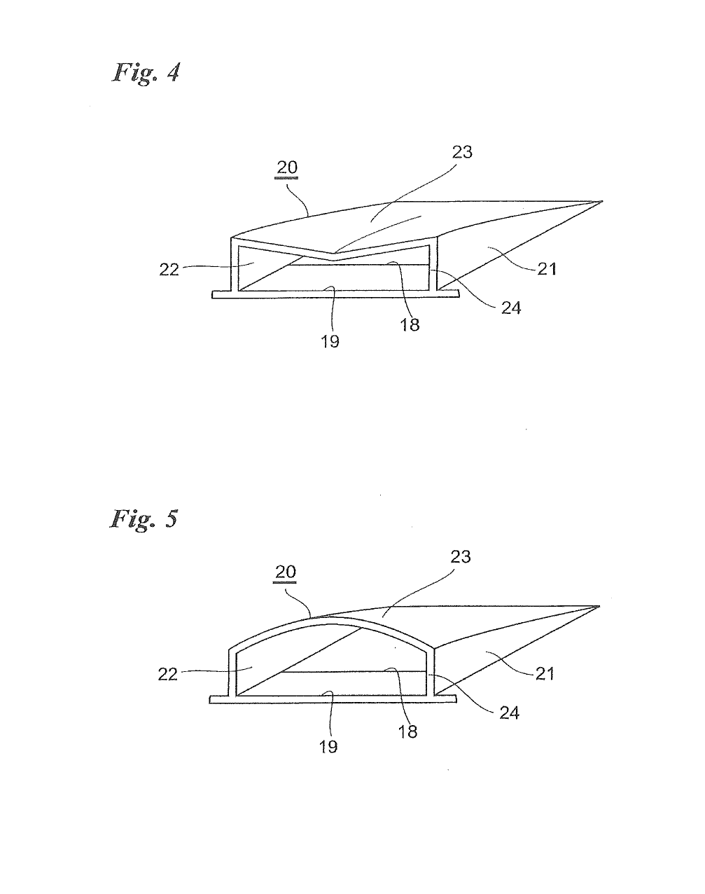

FIG. 4 is a perspective view showing details of the cleaning nozzle 20.

Referring mainly to FIGS. 4 and 3, the cleaning nozzle 20 has an M-shaped end face 24 with an ejection port 19 formed therein (the end face may not necessarily be M-shaped). The ejection port 19 faces the center of the leading end face 16 of the insertion portion 14. The right and left side surfaces 21, 22 of the cleaning nozzle 20 have an approximately triangular shape slanted gently downward toward the outer side of the leading end face 16 of the insertion portion 14. The upper surface 23 of the cleaning nozzle 20 sags in the middle. The ejection port 19 of the cleaning nozzle 20 is connected with the flow path 18 of the rigid-endoscope oversheath 10.

The right and left side surfaces 21, 22 (non-movable portions) of the cleaning nozzle 20 are harder than the upper surface 23 (movable portion) (i.e. the upper surface 23 is softer than the right and left side surfaces 21, 22). For example, the right and left side surfaces 21, 22 are made of polyvinyl chloride (elastic member), while the upper surface 23 is made of styrene-ethylene-butylene-styrene block copolymer (elastic member). The insertion portion 14 may be made of styrene-ethylene-butylene-styrene block copolymer (elastic member) or polyvinyl chloride. It will be understood that the right and left side surfaces 21, 22 and the upper surface 23 of the cleaning nozzle 20 are not necessarily required to adopt respectively different materials as long as the right and left side surfaces 21, 22 are harder than the upper surface 23 (rigid against the fluid). The right and left side surfaces 21, 22 may be thickened using a flexible material, while the upper surface 23 may have a thickness smaller than those of the right and left side surfaces 21, 22. Alternatively, the upper surface 23 may be composed of an elastic member, while the right and left side surfaces 21, 22 may be composed of a non-elastic member.

Fluid such as cleaning solution flows through the flow path 18 of the insertion portion 14 to be ejected from the ejection port 19 of the cleaning nozzle 20. Since the upper surface 23 of the cleaning nozzle 20 is softer than the right and left side surfaces 21, 22 as mentioned above, the upper surface 23 is widened upward in FIG. 4 (undergoes deformation by the fluid) when the fluid is ejected from the ejection port 19.

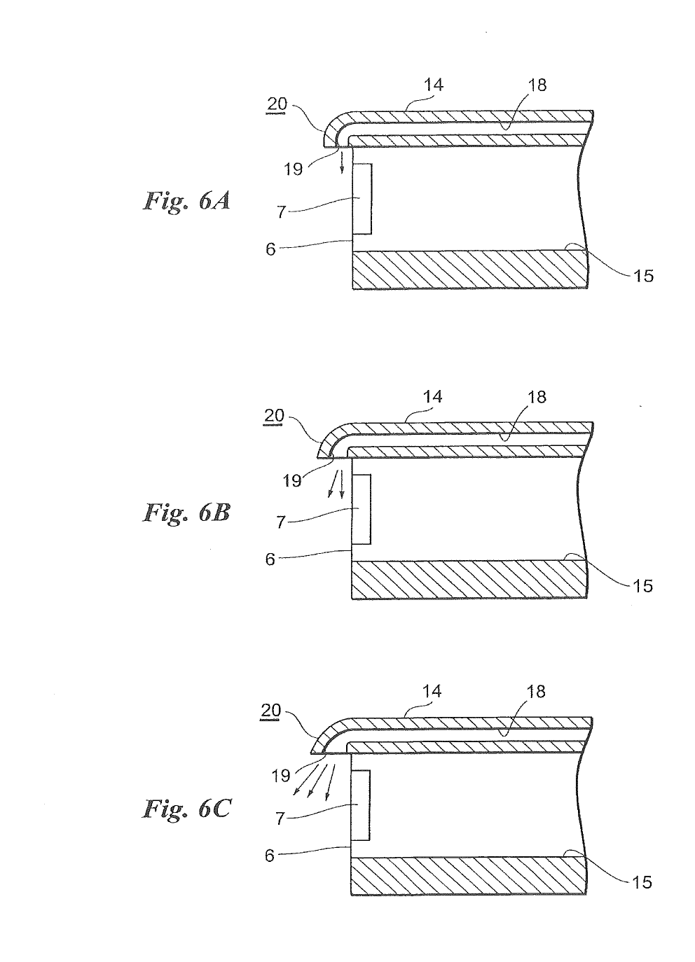

FIG. 5 shows a state of the cleaning nozzle 20 where the upper surface 23 is widened upward.

The fluid ejected from the ejection port 19 brings pressure on the right and left side surfaces 21, 22 and the upper surface 23. Since the upper surface 23 is softer than the right and left side surfaces 21, 22, not the right and left side surfaces 21, 22 but rather the upper surface 23 undergoes deformation. That is, the upper surface 23 is widened upward, namely, the fluid ejected from the ejection port 19 deforms the ejection port 19 itself. The pressure on the right and left side surfaces 21, 22 and the upper surface 23 of the cleaning nozzle 20 varies depending on the amount of ejection of the fluid from the ejection port 19, which causes the ejection port 19 to be deformed according to the flow rate (i.e. the ejection port 19 is not deformed when the flow rate of the fluid is low, while widened when the flow rate of the fluid is high).

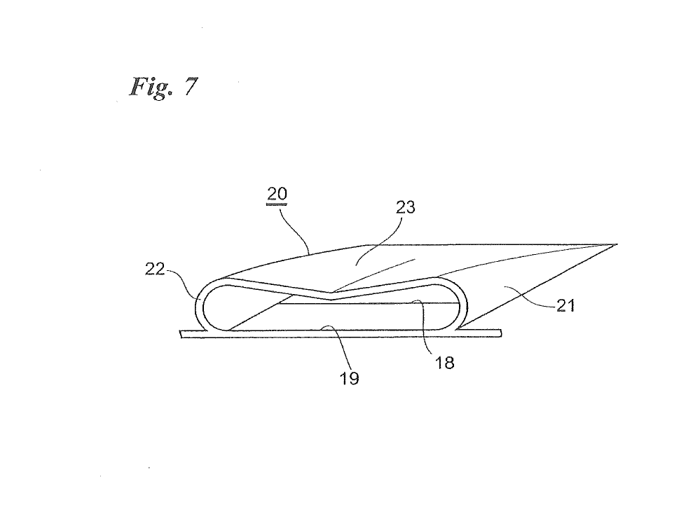

FIGS. 6A to 6C are partial cross-sectional views of the rigid endoscope 1 covered with the rigid-endoscope oversheath 10, corresponding to FIG. 2.

As mentioned above, the ejection port 19 of the cleaning nozzle 20 is deformed according to the flow rate of the fluid ejected from the ejection port 19. FIGS. 6A to 6C show the development of deformation of the ejection port 19 according to the flow rate. The area (second area) of the ejection port 19 is not changed when no fluid is ejected from the ejection port 19, while the area (first area) of the ejection port 19 is greater than the second area when fluid is ejected from the ejection port 19.

FIG. 6A shows a state where the flow rate is low.

When the flow rate of the fluid ejected from the ejection port 19 is low, the pressure on the right and left side surfaces 21, 22 and the upper surface 23, which constitute the cleaning nozzle 20, is also low, resulting in less deformation of the ejection port 19. This causes the fluid to flow in the vicinity of the cover glass 7 as indicated by the arrow. For example, when gas flows through the flow path 18 at a low flow rate, the gas is also ejected from the ejection port 19 at a low flow rate, which causes the gas to flow only in the vicinity of the surface of the cover glass 7, allowing for prevention of condensation due to the difference between the temperature in the body cavity and the surface temperature of the cover glass 7 (gas curtain mode).

FIG. 6B shows a state where the flow rate is a little high.

When fluid is ejected from the ejection port 19 at a little higher flow rate, the pressure on the right and left side surfaces 21, 22 and the upper surface 23, which constitute the cleaning nozzle 20, also increases a little, which causes the upper surface 23 of the cleaning nozzle 20 to be widened a little upward (leftward in FIG. 6B). In FIG. 6B, since the ejection port 19 is deformed and widened leftward, the fluid flows not only in the vicinity of the cover glass 7 but also in a direction a little away from the front surface of the cover glass 7 as indicated by the arrows. For example, cleaning solution flows through the flow path 18 and then reaches not only the upper part but also the lower part of the cover glass 7 in FIG. 6B. This allows the cover glass 7 to be cleaned entirely (lens surface cleaning mode).

FIG. 6C shows a state where the flow rate is high.

When fluid is ejected from the ejection port 19 at a higher flow rate, the pressure on the right and left side surfaces 21, 22 and the upper surface 23, which constitute the cleaning nozzle 20, also increases, which causes the upper surface 23 of the cleaning nozzle 20 to be widened upward (leftward in FIG. 6C) as mentioned above. In FIG. 6C, since the ejection port 19 is deformed and widened leftward, the fluid is ejected not only in the vicinity in front of the cover glass 7 but also forward to a distance a little away from the cover glass 7 as indicated by the arrows. When gas is ejected from the ejection port 19 at a higher flow rate, suspended solids such as smoke and/or mist, if exist in front of the cover glass 7, can be removed therefrom (smoke removal mode). This can prevent suspended solids from attaching to the cover glass 7.

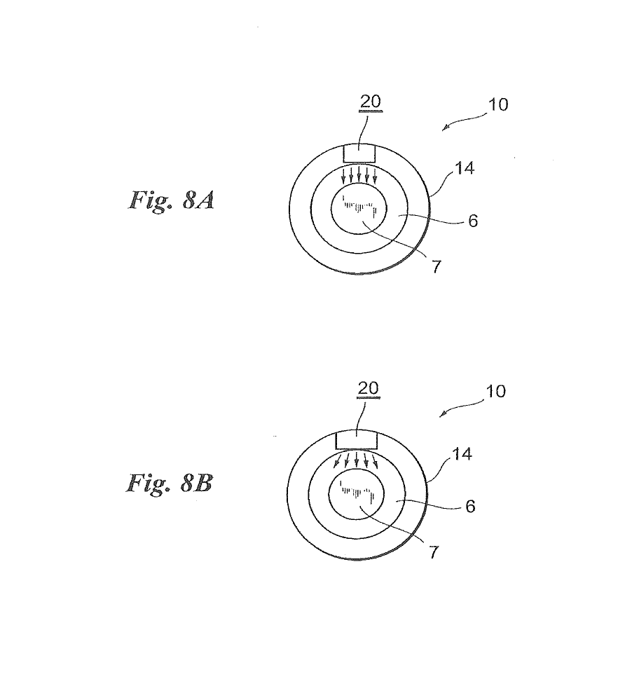

FIG. 7 is a perspective view of a cleaning nozzle 20 according to an exemplary variation, corresponding to FIG. 4.

In the embodiment above, the upper surface 23 of the cleaning nozzle 20 is softer than the right and left side surfaces 21, 22. On the contrary, in this exemplary variation, the right and left side surfaces (movable portions) 21, 22 of the cleaning nozzle 20 are softer than the upper surface 23 (non-movable portion rigid against the fluid). Therefore, when fluid is ejected from the ejection port 19 at a higher flow rate as mentioned above, the right side surface 21 is deformed in such a manner as to expand rightward, while the left side surface 22 is deformed in such a manner as to expand leftward, with the upper surface 23 undergoing less deformation.

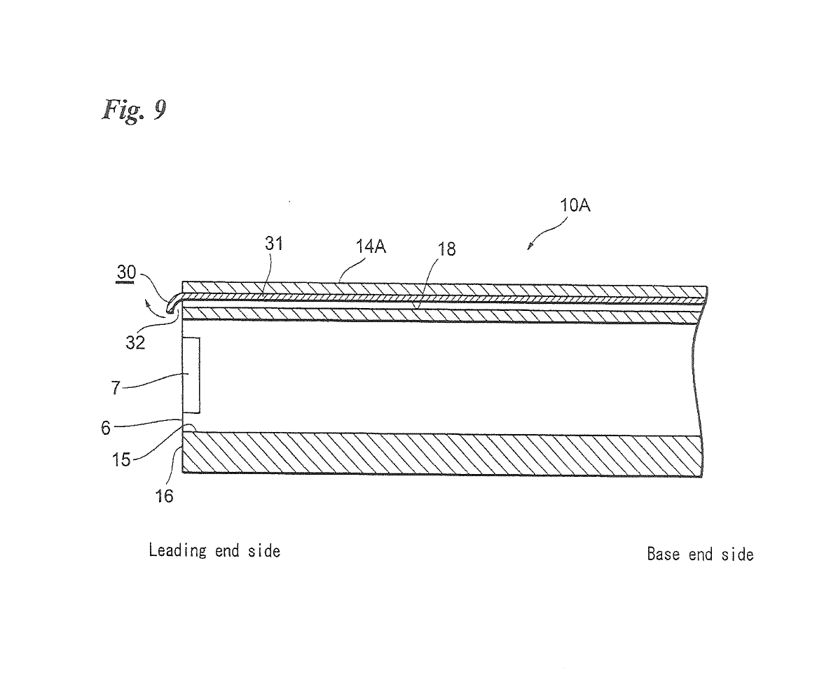

FIGS. 8A and 8B are front views of the rigid endoscope 1 covered with the rigid-endoscope oversheath 10, showing aspects where fluid is ejected from the cleaning nozzle 20 shown in FIG. 7.

FIG. 8A shows a state where the flow rate of the fluid ejected from the cleaning nozzle 20 is low.

When the flow rate is low, the ejection port 19 of the cleaning nozzle 20 undergoes no deformation. Therefore, the fluid is ejected approximately in parallel from the ejection port 19 when viewed from the front of the rigid endoscope 1 (from the front of the cover glass 7) as indicated by the arrows.

FIG. 8B shows a state where the flow rate of the fluid ejected from the cleaning nozzle 20 is high.

When the flow rate is high, the ejection port 19 of the cleaning nozzle 20 is deformed and widened laterally in FIGS. 7 and 8B as mentioned above. Therefore, the fluid is ejected laterally widely from the ejection port 19 as indicated by the arrows.

The direction of ejection of the fluid can thus be changed laterally (in the width direction) by changing the flow rate of the fluid.

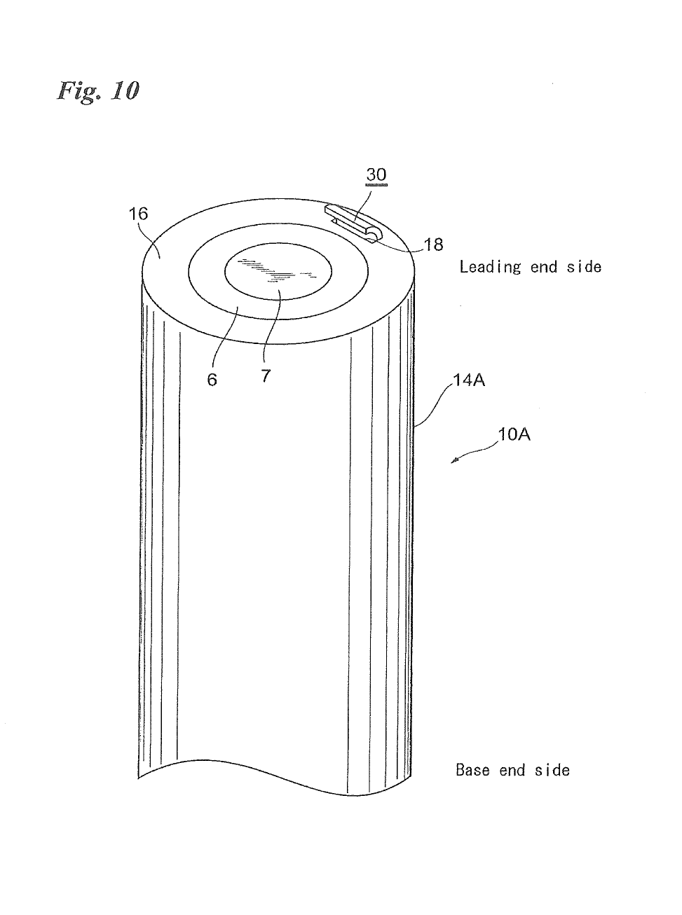

FIGS. 9 and 10 show another exemplary variation. FIG. 9 is a partial cross-sectional view of the rigid endoscope 1 covered with the rigid-endoscope oversheath 10A, corresponding to FIG. 2. FIG. 10 is a perspective view of the rigid endoscope 1 covered with the rigid-endoscope oversheath 10A, corresponding to FIG. 3. In these figures, components identical to those shown in FIGS. 2 and 3 are designated by the same reference numerals to omit the descriptions thereof.

Referring to FIG. 9, on the inner wall of the flow path 18 is provided a restriction plate 31 longitudinally along the flow path 18. The leading end portion 30 of the restriction plate 31 (flexible nozzle, movable portion, or elastic member) extends from the flow path 18 outside the insertion portion 14A of the rigid-endoscope oversheath 10A. The leading end portion 30 is bent inward at the leading end face 16 of the rigid-endoscope oversheath 10. The leading end portion 30 is composed of a flexible material (e.g. styrene-ethylene-butylene-styrene block copolymer mentioned above). The portion of the restriction plate 31 other than the leading end portion 30 may be composed of a flexible material similarly as the leading end portion 30 or the same material or may not be flexible. The insertion portion 14A also may or may not be flexible and, if flexible, may be softer or harder than the leading end portion 30.

In FIG. 9, the space between the leading end portion 30 and the leading end face 16 of the rigid-endoscope oversheath 10A serves as an ejection port 32 from which fluid flowing through the flow path 18 is ejected. Since the leading end portion 30 is bent inward at the leading end face 16 of the rigid-endoscope oversheath 10A, the ejection port 32 faces the cover glass 7 provided on the rigid endoscope 1 when the rigid endoscope 1 is covered with the rigid-endoscope oversheath 10A. Thus, the fluid ejected from the ejection port 32 is guided over the surface of the cover glass 7.

Since the leading end portion 30 constituting the ejection port 32 is flexible, fluid, when ejected from the ejection port 32 at a higher flow rate, opens the leading end portion 30 to widen the ejection port 32 as indicated by the arrow in FIG. 9. The fluid ejected from the ejection port 32 therefore reaches not only the vicinity of the front surface of the cover glass 7 but also a distance away from the cover glass 7. When the fluid is ejected from the ejection port 32 at a lower flow rate, the leading end portion 30 stays unmoved and the ejection port 32 undergoes no deformation (to have the second area). The fluid ejected from the ejection port 32 therefore does not reach a distance away from the cover glass 7 but only flows in the vicinity of the front surface of the cover glass 7. Also in this exemplary variation shown in FIGS. 9 and 10, the ejection port 32 is deformed according to the flow rate of the fluid (to have the first area) and thereby the direction of ejection of the fluid also changes.

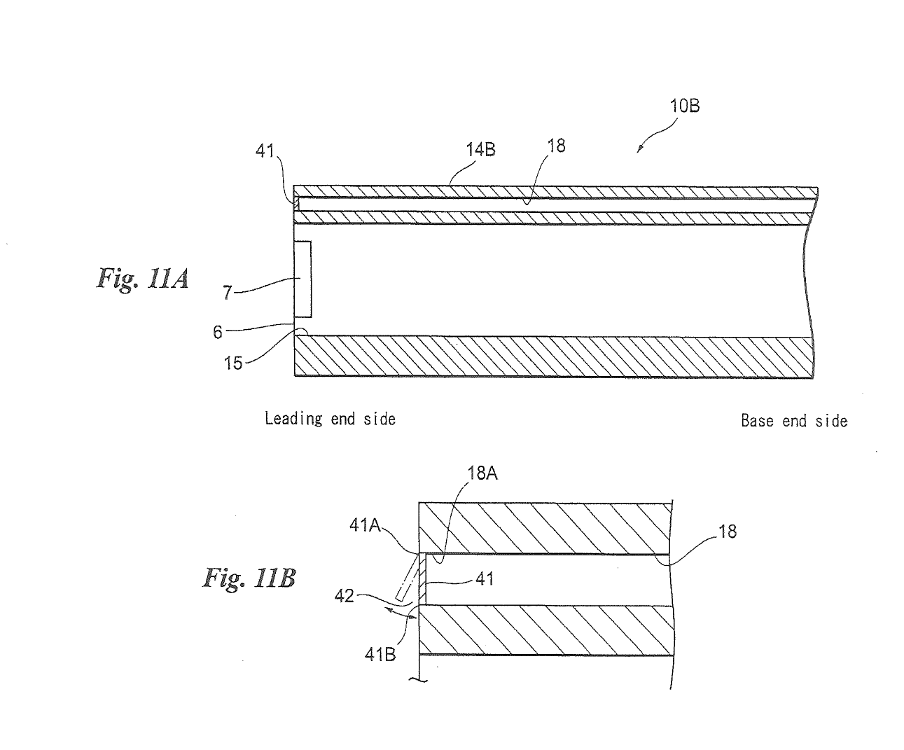

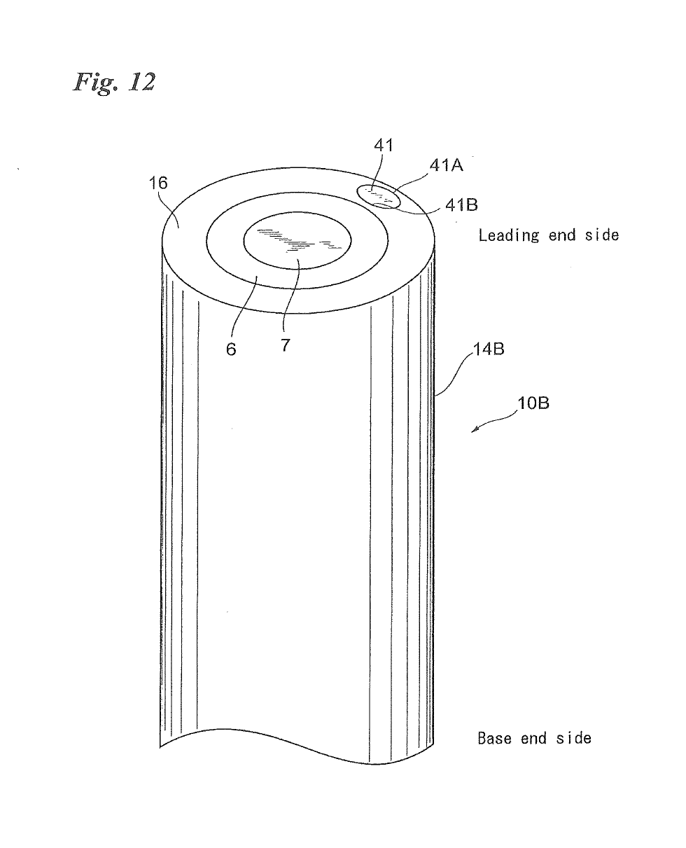

FIGS. 11A, 11B, and 12 show still another exemplary variation. In these figures, components identical to those described above are designated by the same reference numerals to omit the descriptions thereof.

FIG. 11A shows an aspect where the insertion portion 5 of the rigid endoscope 1 is covered with the insertion portion 14B of the rigid-endoscope oversheath 10B, corresponding to FIG. 2. FIG. 11B is an enlarged view of the leading end portion shown in FIG. 11A. FIG. 12 is a perspective view of the leading end portion of the rigid endoscope 1 covered with the rigid-endoscope oversheath 10B, corresponding to FIG. 3.

At the leading end of the flow path 18 is formed a valve 41 having the same shape (circular) as the cross-section of the flow path 18. The valve 41 may or may not be flexible. One outer end portion 41A on the peripheral surface of the valve 41 is composed of an elastic member (movable portion) and fixed to a portion of the inner wall surface 18A on the outer side of the flow path 18. The portion 41B other than the one end portion 41A is not fixed to the inner wall surface of the flow path 18 to serve as a free end. As shown in FIG. 11B, the valve 41 closes the leading end of the flow path 18 openably using the one end portion 41A as a support at the leading end of the flow path 18. The leading end of the flow path 18 serves as an ejection port 42 (second area).

Fluid flows through the flow path 18 and then presses the valve 41 as indicated by the dot-dashed line, which widens the ejection port 42 as shown in FIG. 11B. The fluid is then ejected from the ejection port 42. When the flow rate of the fluid is low, the valve 41 is less pressed and therefore less opened to result in the ejection port 42 staying narrow. The fluid ejected from the ejection port 42 is provided to the vicinity in front of the cover glass 7. When the flow rate of the fluid is high, the valve 41 is applied with a greater force and therefore more opened to result in the ejection port 42 being widened (first area). The fluid ejected from the ejection port 42 reaches not only the vicinity in front of the cover glass 7 but also a distance away from the cover glass 7. The ejection port 42 is deformed according to the flow rate of the fluid and thereby the direction of ejection of the fluid from the ejection port 42 also changes.

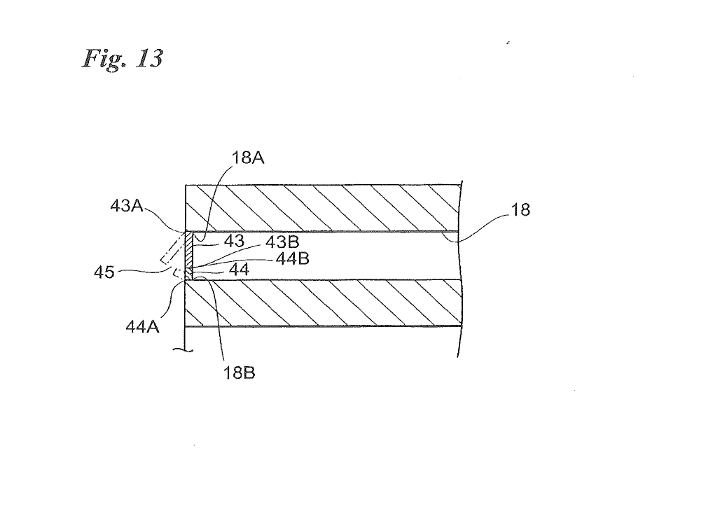

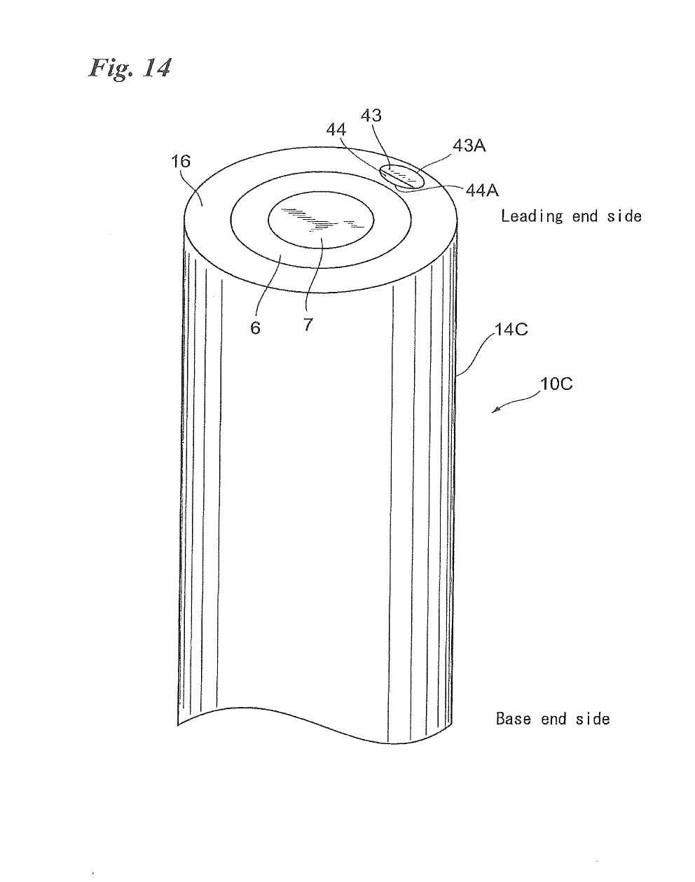

FIG. 13 is a cross-sectional view of the leading end portion of the flow path 18 according to a further exemplary embodiment, corresponding to FIG. 11B. FIG. 14 is a perspective view of the leading end portion of the rigid endoscope 1 covered with the rigid-endoscope oversheath 10C, corresponding to FIG. 3.

In the embodiment above, one valve 41 is formed at the leading end of the flow path 18. In this embodiment shown in FIG. 13, two valves 43, 44 are formed at the leading end of the flow path 18.

The first valve 43 (movable portion) is fixed via one end portion 43A composed of an elastic member to the inner wall 18A on the outer peripheral side at the leading end of the flow path 18 within the insertion portion 14C, while the other end portion 43B is opened. The second valve 44 (movable portion) is fixed via one end portion 44A composed of an elastic member to the inner wall 18B on the inner peripheral side at the leading end of the flow path 18, while the other end portion 44B is opened. The first and second valves 43, 44 have their respective approximately semicircular shapes (see FIG. 14), the combination of the first and second valves 43, 44 having a circular shape capable of closing the leading end of the flow path 18. The first and second valves 43, 44 may or may not be flexible. If both the first and second valves 43, 44 are flexible, portions of the periphery of the first and second valves 43, 44 in contact with the flow path 18 may be fixed to the flow path 18, while the other end portions 43B, 44B of the first and second valves 43, 44 may be in separable contact with each other. This allows fluid to be ejected from between the other end portions 43B, 44B of the first and second valves 43, 44.

Fluid flows through the flow path 18 and then presses and opens the first and second valves 43, 44 to result in the ejection port 45 being widened (first area). The fluid flowing through the flow path 18 is then ejected from the ejection port 45. When the flow rate of the fluid is high, the ejection port 45 is widened and thereby the direction of ejection of the fluid also changes similarly as mentioned above.

If both the first and second valves 43, 44 are flexible, the first valve 43 is softer than the second valve 44. This causes the first valve 43 to be deformed more outward (toward the leading end) than the second valve 44, whereby the fluid is ejected toward the center of the rigid-endoscope oversheath 10C (i.e. toward the cover glass 7). On the contrary, if the first and second valves 43, 44 are not flexible, one end portion 43A of the first valve 43 composed of an elastic member is softer than one end portion 44A of the second valve 44 composed of an elastic member. Also in this case, the first valve 43 is deformed more outward than the second valve 44. One of the first and second valves 43, 44 may be flexible, while the other may not be flexible.

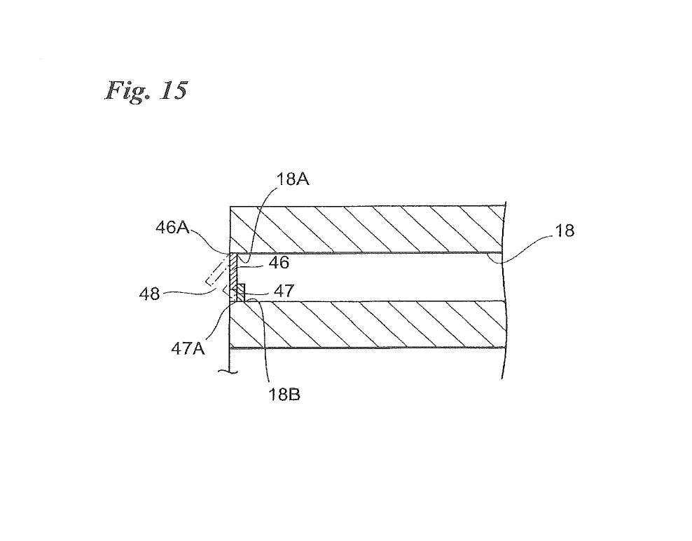

FIG. 15 is a cross-sectional view of the leading end portion of the flow path 18 according to a still further exemplary embodiment, corresponding to FIG. 13.

One end portion 46A (elastic member) of the first valve 46 is fixed to the inner wall 18A on the outer side at the leading end of the flow path 18, while one end portion 47A (elastic member) of the second valve 47 is fixed to the inner wall 18B on the inner side at the leading end of the flow path 18 similarly as mentioned above. Unlike the first and second valves 43, 44 shown in FIGS. 13 and 14, the first and second valves 46, 47 shown in FIG. 15 are provided at longitudinally different positions in the flow path 18.

When no fluid flows through the flow path 18, the first and second valves 46, 47 close the leading end of the flow path 18. When fluid flows through the flow path 18, the first and second valves 46, 47 are opened to provide an ejection port 48. When no fluid flows through the flow path 18, the ejection port 48 is closed. Also in this exemplary variation, the ejection port 48 is deformed according to the flow rate of the fluid and thereby the direction of ejection of the fluid from the ejection port 48 also changes.

In the embodiments above, all the first valves 43, 46 and the second valves 44, 47 may or may not be flexible. In addition, the first valves 43, 46 may be softer than the second valves 44, 47. When the fluid flows, the first valves 43, 46 are more open than the second valves 44, 47, whereby the direction of ejection of the fluid from the ejection ports 45, 48 can be adjusted.

In the embodiments above, when no fluid flows through the flow path 18 (i.e. no fluid is ejected from the ejection port 42), the leading end of the flow path 18 is closed. Therefore, even when the rigid endoscope 1 is inserted into a body cavity expanded by carbon dioxide and the like, it is possible to prevent carbon dioxide filling the body cavity from counterflowing into the flow path 18. It is preferable to form a stopper in the flow path 18 to prevent the valve 41 shown in FIGS. 11A and 11B, the first and second valves 43, 44 shown in FIG. 13, and the first and second valves 46, 47 shown in FIG. 15 from being opened toward the interior of the flow path 18.

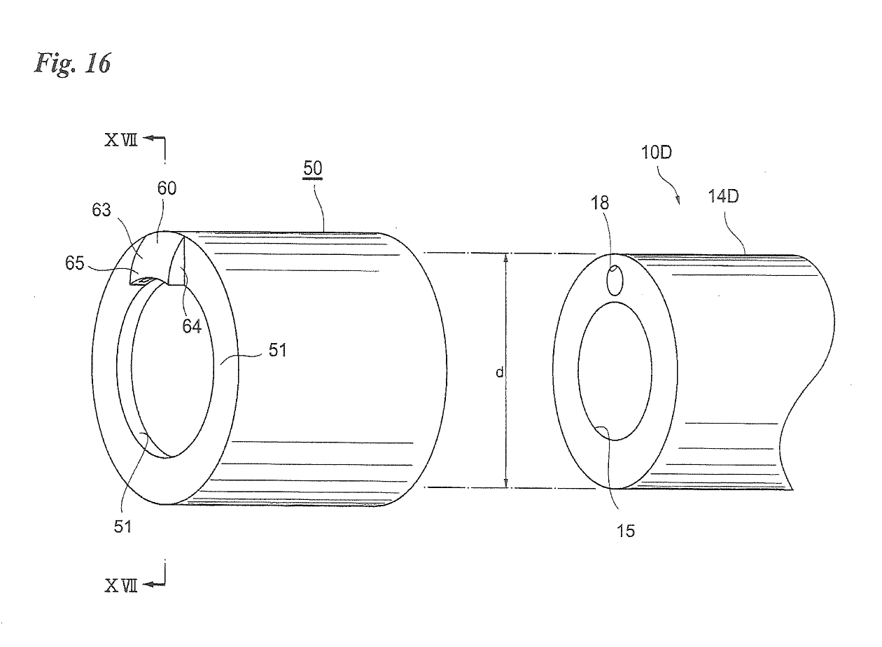

FIGS. 16 to 18 show another embodiment.

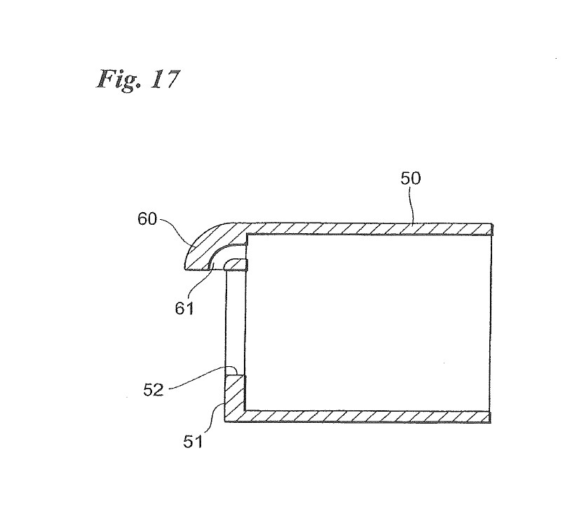

FIG. 16 is a perspective view showing a leading end portion of an insertion portion 14D of a rigid-endoscope oversheath 10D and a cap 50 put on the leading end portion of the insertion portion 14D. FIG. 17 is a cross-sectional view of the cap 50 taken along the line XVII-XVII of FIG. 16.

In the embodiments shown in FIG. 1, for example, the cleaning nozzle 20 is formed in the rigid-endoscope oversheath 10 itself. In this embodiment, a cleaning nozzle 60 (flexible nozzle, elastic member, or movable portion) is formed at the leading end face 51 of the cap 50 to be put on the rigid-endoscope oversheath 10D.

In the leading end face 51 of the cap 50 is formed an opening 52. When the rigid endoscope 1 is covered with the rigid-endoscope oversheath 10D on which the cap 50 is put, the cover glass 52 provided at the leading end of the rigid endoscope 1 can be seen through the opening 52.

As shown in FIG. 17, the cleaning nozzle 60 formed in the cap 50 is formed with a flow path 61 for ejecting fluid flowing through the flow path 18, which is formed in the rigid-endoscope oversheath 10D as mentioned above, from the cleaning nozzle 60. The flow path 61 is bent at approximately 90 degrees so that the ejection port 62 faces the opening 52. The cleaning nozzle 60 includes a relatively soft upper surface 63 and relatively hard side surfaces 64, 65, as is the case with the above-described cleaning nozzle 20 (see FIG. 4).

The cap 50 has a circular tubular shape with an inside diameter of “d” approximately the same as the outside diameter of the insertion portion 14D of the rigid-endoscope oversheath 10D.

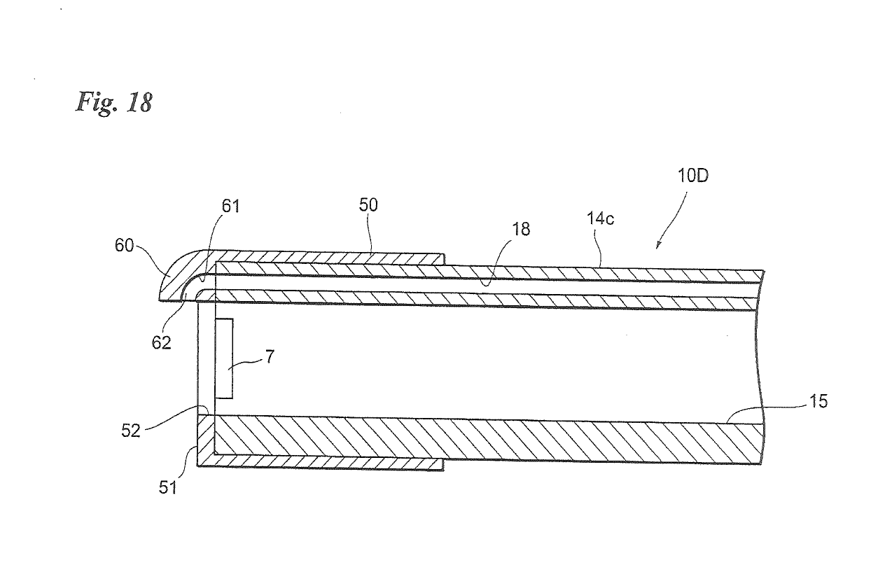

FIG. 18 is a cross-sectional view of the insertion portion 5 of the rigid endoscope 1 covered with the insertion portion 14D of the rigid-endoscope oversheath 10D with the cap 50 put thereon, corresponding to FIG. 2.

As shown in FIG. 18, when the cap 50 is fitted onto the rigid-endoscope oversheath 10D such that the flow path 18 formed in the rigid-endoscope oversheath 10D is aligned with the flow path 61 formed in the cap 50, the flow path 18 formed in the rigid-endoscope oversheath 10D comes into communication with the flow path 61 formed in the cap 50. Fluid flows through the rigid-endoscope oversheath 10D and then the flow path 61 in the cleaning nozzle 60 of the cap 50 to be ejected from the ejection port 62.

Since the cleaning nozzle 60 includes the relatively soft upper surface 63 and relatively hard side surfaces 64, 65, the ejection port 62 is deformed according to the flow rate of the fluid flowing through the ejection port 62 (see FIG. 5). The area (first area) of the ejection port 62 when the fluid is ejected therefrom is greater than the area (second area) of the ejection port 62 when no fluid is ejected therefrom. As mentioned above, the direction of ejection of the fluid can be changed according to the flow rate of the fluid.

Although the upper surface 63 of the cleaning nozzle 60 is relatively soft while the side surfaces 64, 65 are relatively hard in the embodiment above, the upper surface 63 may be relatively hard while the side surfaces 64, 65 may be relatively soft. As shown in FIG. 7, this causes the side surfaces 64, 65 to be widened according to the flow rate of the fluid, whereby the direction of ejection can be changed such that the fluid is ejected laterally widely from the ejection port 62 (see FIG. 8B).

Although in the embodiment above, the cleaning nozzle 60 formed in the cap 50 is of the same kind as the cleaning nozzle 20 shown in FIGS. 1 to 8A, 8B, not such a cleaning nozzle 60 but, for example, a leading end portion 30 shown in FIGS. 9 and 10 or first and second valves 41, 42 shown in FIGS. 11A, 11B to 13 may be formed in the flow path 61.

Although in the embodiment above, the cap 50 is fitted onto the leading end of the insertion portion of the rigid-endoscope oversheath 10D, not such a fitting type but another type of fixation may be employed such as fixing the rear end face of the cap 50 adhesively, threadably, or weldedly to the leading end face of the rigid-endoscope oversheath 10D.

Although in the embodiment above, the cleaning nozzle 60 is formed in the cap 50 to be put on the rigid-endoscope oversheath 10D, such a cleaning nozzle 60 may be formed similarly in a cap to be put on the rigid endoscope 1 itself. Such a cap may serve as the above-described rigid-endoscope oversheath 10, for example.

What is claimed is:

1. A rigid-endoscope oversheath having a leading end, a base end, a longitudinal shaft, and a lumen into which a rigid endoscope can be inserted, the oversheath comprising:
a flow path through which fluid flows from the base end toward the leading end along the longitudinal shaft;
an ejection port formed in a same plane with a leading end plane of the rigid-endoscope oversheath to eject the fluid flowing through the flow path from the leading end plane of the flow path, a peripheral portion of the port being at least partially composed of an elastic member deformable by the fluid flowing through the flow path; and
a valve at least partially attached to a leading end of the flow path to openably close the leading end of the flow path.

2. The rigid-endoscope oversheath according to claim 1, further comprising a valve at least partially attached to a leading end of the flow path to openably close the leading end of the flow path.

3. The rigid-endoscope oversheath according to claim 2, wherein
a portion through which the valve is attached to the leading end of the flow path or the valve itself is composed of the elastic member.

4. The rigid-endoscope oversheath according to claim 2, wherein
the valve comprises first and second valves with one end thereof being attached to a wall surface of the flow path at the leading end of the flow path, and wherein
the first and second valves are configured to openably close the leading end of the flow path.

5. The rigid-endoscope oversheath according to claim 4, wherein
the first valve is provided on the outer peripheral side at a leading end face of the rigid-endoscope oversheath, while the second valve is provided on the inner peripheral side at the leading end face of the rigid-endoscope oversheath, and wherein
the first valve is softer than the second valve.

6. The rigid-endoscope oversheath according to claim 1, further comprising a cap to be put on the leading end of the rigid-endoscope oversheath, wherein
the ejection port is formed in the cap.

7. The rigid-endoscope oversheath according to claim 1, wherein said valve has a same shape as a cross-section of said flow path.

8. The rigid-endoscope oversheath according to claim 1, wherein said valve is pushed by the fluid when the fluid flows through said flow path.

9. The rigid-endoscope oversheath according to claim 1, wherein said valve is flexible.

10. The rigid-endoscope oversheath according to claim 1, wherein said valve includes an outer end portion on a peripheral surface of the valve,
wherein the outer end portion comprises an elastic member, and
wherein the outer end portion is fixed to an inner wall surface on an outer side of the flow path.

11. The rigid-endoscope oversheath according to claim 10, wherein said valve includes a second portion which is not fixed to the inner wall surface of the flow path, and
wherein the second portion of said valve serves as a free end.

12. The rigid-endoscope oversheath according to claim 1, further comprising a plurality of valves at least partially attached to the leading end of the flow path to openably close the leading end of the flow path.
